(19) United States
(12) Patent Application Publication
(10) Pub. No.: US 2013/0195988 A1
(43) Pub. Date: Aug. 1, 2013

(54) VASCULAR EMBOLIZATION GELLING AGENT FOR SUSTAINED RELEASE OF DRUGS FOR TREATING TUMORS AND METHOD FOR PREPARING THE SAME

(71) Applicant: Shanghai Cancer Institute, Shanghai (CN)

(72) Inventors: Yourong DUAN, Shanghai (CN); Zhiyong WAN, Shanghai (CN); Ming SHEN, Shanghai (CN); Qiufen SUN, Shanghai (CN)

(73) Assignee: Shanghai Cancer Institute, Shanghai (CN)

(21) Appl. No.: 13/791,629

(22) Filed: Mar. 8, 2013

Related U.S. Application Data
(63) Continuation of application No. PCT/CN2011/001510, filed on Sep. 6, 2011.

(30) Foreign Application Priority Data
Sep. 8, 2010 (CN) 201010275424.0
Apr. 8, 2011 (CN) 201110088056.3

(51) Int. Cl. A61K 9/50

(52) U.S. Cl. CPC A61K 9/5031; A61K 9/5026
USPC 424/497; 424/490; 514/616; 514/17.2; 514/34; 514/252.18; 514/492; 424/649; 514/346

(57) ABSTRACT

A vascular embolization gelling agent for sustained release of drugs for treating tumors having a drug and a drug carrier. The drugs are antitumor drugs. The drug carrier includes poloxamer polymer and polyvinylpyrrolidone or gel made of the combination, and may be purified before use. The drug carrier accounts for 5-65% of the gel. The particle size of the gel is in the range of 10 nm-150 μm. The embolization agent is a liquid gel at normal temperature, to facilitate direct transcatheter injection, and is rapidly solidified to the gel state in body with the increase of the temperature; it is used to encapsulate different drugs on demand, and can achieve dual efficacy of embolization and drug treatment through local sustained release of the drug. The present invention can be used as the embolization agent for endovascular interventional therapy for transcatheter arterial chemoembolization of various benign and malignant tumors.

### CROSS-REFERENCE AND RELATED APPLICATIONS

The subject application is a continuation of PCT international application PCT/CN2011/001510 filed on Sep. 6, 2011, which in turn claims priority on Chinese patent application Nos. CN 201010275424.0 filed on Sep. 8, 2010 and CN201110088056.3 filed on Apr. 8, 2011. The contents and subject matter of all the PCT and Chinese priority applications are incorporated herein by reference.

### TECHNICAL FIELD

The present invention relates to pharmaceutical preparations, specifically relates to the drugs encapsulated with gel carriers, and establishes a thermosensitive gel for sustained release of vascular embolization agent, especially relates to a vascular embolization gelling agent used for sustained release of drugs for the treatment of tumor and the preparation method thereof.

### BACKGROUND TECHNOLOGY

At present, the interventional therapy is one of the more popular and effective methods used to treat advanced cancers. In the interventional therapy, transcatheter arterial infusion (TAI) chemotherapy and transcatheter arterial chemoembolization (TACE) are most frequently used. A lot of experimental and clinical studies (HinoT, Kawashima Y, Shimabayashi S. Basic study for stabilization of W/O/W emulsion and its application to transcatheter arterial embolization therapy [J]. Adv Drug Deliv Rev, 2000, 45 (1), 27-45) proved that the implementation of TAI by transcatheter choice or superselective insertion into the feeding arteries of tumors can significantly improve the local drug concentration in tumors, prolong the contact time of drug, and improve the drug efficacy. But usually, the patients simply treated with the TAI method have low survival rate in five years, and the long-term efficacy of the TAI method is not more significantly improved than that of the intravenous chemotherapy; in addition, TAI is still limited to the dose of chemotherapeutic drugs, and is affected by the drug resistance of tumor and cancer cell proliferation; TAI is a short-time high-concentration implosive therapy for tumor with limited local action time of drugs and minor efficacy on tumor cells in systemic blood and distant metastasis, and its long-term efficacy is not satisfactory, either. The TACE method can achieve the purpose of tumoral ischemia and hypoxia-induced necrosis by blocking blood supply to tumors, and the necrotic cancer tissues can stimulate the body immunity, and may remove distant metastases; injection of mixed embolization agent and chemotherapeutic drugs into the target feeding arteries of tumors can not only block the blood supply, but also slowly release chemotherapeutic drugs, and play a role in local chemotherapy. Therefore, the short-term efficacy of TACE is better than that of TAI. However, TACE has the weakness such as drug resistance of tumors and repeated embolization required for tumor angiogenesis. Some literatures reported that after embolization treatment, local hypoxia has the effect of inducing the expression of VGEF and promoting tumor angiogenesis (Wang Bin, Xu Hui and Cao Guiwen et al. The effect of transcatheter arterial chemoembolization on angiogenesis and the expression of vascular endothelial growth factor in hepatocellular carcinoma. Chinese Journal of Radiology, 2005, 39 (2): 204-206). However, its long-term efficacy is still not satisfactory. Therefore, investigation of the embolization agent for further improving the long-term efficacy of interventional therapy of tumors becomes one of the most important subjects in medical researches of interventional therapy, and the embolization material thereof is crucial.

At present, the embolization materials used in clinical and experimental studies mainly include: (1) inactive materials, autologous materials and radioactive particles according to the material properties; (2) solid and liquid embolization materials according to the physical properties; (3) short-term, medium-term and long-term embolization materials according to the duration of the vascular occlusion; (4) absorbable and non-absorbable embolization materials according to the absorbability to the body. Liquid embolization agent widely used in clinical treatment with TACE is iodipin, which is mainly used in embolization treatment of liver cancer and other malignant tumors, and is generally made into suspension or emulsion by mixing with chemotherapeutic drugs. Its action principle is as follows: iodipin is directly injected into the feeding arteries of tumor through catheter, is deposited in tiny arteries and blood sinus of tumor, plays a role in embolizing the peripheral arteries of tumor and sustained release, and prolongs the local chemotherapy time because it contains chemotherapeutic drugs. But many clinical and experimental studies showed that iodipin can be only used to fill in peripheral vessels, is not a desired embolization agent, and can be lost with the vascular recanalization; liquid embolization agent, whether suspension or emulsion, has short time for sustained release, which is generally within 24-72 hours. Solid embolization agent, such as gelatin foam microspheres and PVA (polyvinyl alcohol) etc., cannot be deformed with the size and shape of vessels, and has the weakness such as incomplete embolization, induction of tumor angiogenesis and collateral vessel formation. In other reports of bletilla microsphere embolization agent, bletilla has the effect of both mechanical obstruction and strong procoagulation, and therefore, its clinical efficacy is more reliable than that of other embolization agents, but at present, it is still not promoted in clinical application because of severe side reactions such as pain, fervescence, and liver function damage etc. after embolization with it. Besides, there are insufficient clinical and experimental researches of albumin, starch, cisplatin bletilla striata gum, polylactic acid, chitosan and other microspheres. Thus, at present, there are many embolization agents in clinical and experimental studies, but research and improvement of them are still necessary.

### SUMMARY OF THE INVENTION

Technical matters to be solved in the present invention are to overcome the weakness of embolization agents in clinical applicaiton, select appropriate well-biocompatible polymer as the carrier for encapsulation of drugs, and establish a thermosensitive gel for sustained release of embolization agent, which is liquid at room temperature and gel at body temperature, so as to achieve the effect of embolization and purpose of sustained release of drugs.

The present invention provides a vascular embolization gelling agent used for sustained release of drugs for the treatment of tumors.

The vascular embolization gelling agent used for sustained release of drugs for the treatment of tumors defined in the present invention is prepared through encapsulation with the gel made of drug carriers; drug content in the vascular embolization gelling agent is in the range of 0.01%-50% W/W, and is preferably 0.5-50% W/W.

Mass percentage of the drug carrier in the vascular embolization gelling agent is 10%-65%.

The drug carrier can be purified by recrystallization before preparation of the embolization agent.

The gel particle size of the gelling agent is in the range of 10 nm-150 μm, and is preferably 100 nm-50 μm.

The drug carrier in the present invention is one or more of polymer poloxamer or synthetic polymers such as polyvinylpyrrolidone (PVP), polyethylene glycol (PEG), polyvinyl alcohol (PVA), carbomer, polymethacrylates, PEG-modified polylactic acid (PEG-PLA), PEG-modified polylactic acid-glycolic acid (PEG-PLGA), PEG-modified polyglycolic acid (PEG-PGA), PEG-modified polycaprolactone (PEG-PCL); cellulose such as methyl cellulose (MC), ethyl cellulose (EC), hydroxyethyl cellulose (HEC), hydroxyethyl methyl cellulose (MHEC), hydoxymethyl cellulose (HMC), hydroxypropyl methyl cellulose (HPMC), hydroxypropyl cellulose (HPC), carboxymethyl cellulose (CMC), sodium carboxymethyl cellulose (CMC-Na); modified starches such as pregelatinized starch; natural gums such as gum acacia, tragacanth gum, carrageenan, locust bean gum, guar gum, konjac gum, alginate, gelatin, hyaluronic acid and agar; non-cellulosic polysaccharides such as chitosan, deacetylized chitosan, galactomannan, cyclodextrin and cyclodextrin derivatives. The molecular weight of each polymer in the drug carrier is 0.1K-5000K Dalton, and is preferably 0.5K-50K Dalton.

The drug carrier in the present invention is preferably poloxamer (main part) with or without addition of one or more of the above polymers, and is most preferably the combination of the main part poloxamer 407 (Pluronic® F127, Lutrol® F127) with one or more of other polymers. Poloxamer covers 100%-0.1% of the carrier material, and one or more of the above added polymers accounts for 0-20% of the carrier material.

The polymer poloxamer in the present invention is also known as pluronic, poloxalkol; monolan; supronic; polyvethylene propylene glycol and pluronic, and includes a series of products. (Chinese Pharmacopoeia 5th Edition) The molecular formula of poloxamer is HO(C₂H₄O)ₐ(C₃H₆O)_{b}(C₂H₄O)ₐH, and its structural formula is:

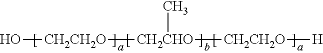

Where, b is 15-67, a is 2-130, and the total proportion of a unit accounts for 20-90 wt % of poloxamer. The molecular weight of poloxamer is preferably in the range of 1000-20000. The type and physicochemical parameters of the poloxamer in the present invention are shown in Table 1. (Note: poloxamer and pluronic are different translations of the same material.)

TABLE 1
Type And Physicochemical Parameters Of Poloxamer

| | Corresponding to | Average | | | | |
|---|---|---|---|---|---|---|
| Poloxamer | the type of | molecular | Melting | Viscosity | | Relative |
| type | poloxamer | weight | point (° C.) | (° C.) | HLB | density |
| 408 | F128 | 12600 | 58 | 3100 | 22 | 1.08 |
| 407 | F127 | 12220 | 58 | 3600 | 21.5 | 1.07 |
| 338 | F108 | 14600 | 57 | 2800 | 27 | 1.07 |
| 333 | P103 | 5800 | 57 | 500 (60° C.) | 12.5 | 1.03 |
| 334 | P104 | 5850 | 37.5 | 550 | 13 | 1.04 |
| 237 | F87 | 7700 | 49 | 700 | 24 | 1.04 |
| 235 | P85 | 4250 | 32 | 265 | 13.8 | 1.05 |
| 215 | P75 | 4150 | 34 | 250 | 16.5 | 1.06 |
| 188 | F68 | 8350 | 52 | 1000 | 29 | 1.06 |
| 108 | F38 | 5000 | 48 | 260 | 30.5 | 1.07 |

* F represents solid (sheet), P represents semisolid (paste).

Physicochemical parameters of preferable poloxamer type:

Melting point of the lowest gel (mass concentration is respectively 60%, 65%, 30% and 20%) of poloxamer 188, 237, 338 and 407 is respectively 52° C., 49° C., 57° C., 56° C. at room temperature, and its viscosity (>7° C., mPa.$) is respectively 100, 700, 2800 and 3100, so poloxamer 407 (Pluronic® F127, Lutrol® F127), poloxamer 338 (Pluronic® F108) and poloxamer 188 (Pluronic® F68, Lutrol® F68) are preferable, and poloxamer 407 (Pluronic® F127) is most preferable.

The generic name of the polyvinylpyrrolidone in the present invention is povidone, it is also called E1201; Kollidon; Plasdone; Polyvidone; poly[1-(2-oxo-1-pyrro-lidinyl) ethylene]; PVP; polyvinylpyrrolidone; 1-vinyl-2-pyrro-lidinone polymer, and its chemical name is 1-vinyl-2-pyrrolidone homopolymer.

(Chinese Pharmacopoeia 5th Edition) Its chemical structural formula is:

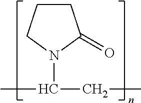

The molecular weight of PVP is characterized by the viscosity of aqueous solution of povidone relative to that of water, and is expressed as the K value in the range of 10-120.

TABLE 2
Approximate Molecular Weight Of Povidone In Various Levels

| K value | Molecular weight |
|---|---|
| 12 | 2500 |
| 15 | 8000 |
| 17 | 10 000 |
| 25 | 30 000 |
| 30 | 50 000 |
| 60 | 400 000 |
| 90 | 1 000 000 |
| 120 | 3 000 000 |

K value of the polyvinylpyrrolidone in the present invention is preferably 15-60, and is most preferably K25, K30 and K90.

The poloxamer and polyvinylpyrrolidone are available on the market, such as poloxamer 407 and poloxamer sold in the name of Pluronic® F127 (BASF Company) or Synperonic PE/F127 (Uniqema).

In the present invention, the term “thermosensitive gel” means that the gel exists in the form of flowable liquid at sufficient concentration and below its critical temperature, becomes gel at a temperature higher than its critical temperature, and reversibly becomes flowable liquid if the temperature drops to below its critical point again.

Another purpose of the present invention is to provide a method for preparation of the vascular embolization gelling agent used for sustained release of drugs for the treatment of tumors through encapsulation with the gel made of drug carriers.

Gelling agent in the present invention can be prepared with a purified or unpurified polymer purchased on the market, and is more preferably prepared with a polymer purified through recrystallization, separation of polymers and then drying.

Poloxamer can be purified with one of the following methods: poloxamer is dissolved in a suitable solvent, such as one or a mixture of more of ethanol, isopropanol, chloroform, dichloromethane and water, and then precipitated in ether or alkane such as ethyl ether or n-hexane. Or it is purified through lay separation in n-propanol/water solvent, and then drying the separated polymer.

Povidone can be purified with one of the following methods: PVP is dissolved in a suitable solvent, such as one or a mixture of more of methanol, ethanol, isopropanol, acetone, chloroform, dichloromethane, ethyl acetate and water, and then precipitated in ether or alkane such as ethyl ether or n-hexane, followed by drying the separated polymer.

Drying method in the present invention may be one or more of rotary evaporation under reduced pressure, drying under reduced pressure, vacuum drying, freeze drying, spray drying, fluidized bed granulation drying and heat drying.

The preparation method for vascular embolization gelling agent used for sustained release of drugs for the treatment of tumors in the present invention comprises the following steps:

(1) Preparation of the Gelling Agent

One or a mixture of more of the carrier materials are added to the solution, which is dispersed while oscillateing, and then laid aside at the temperature of 0-300° C., preferably at the temperature of 0-70° C., and most preferably at the temperature of 0-25° C., until the carrier materials are completely swelled, and dispersed and dissolved into a transparent liquid while stirring. Subsequently, a liquid gelling agent can be obtained by sterilization, aseptic filling and sealing; or a dried gelling agent is obtained through drying, and is preserved at the temperature of −70° C.˜50° C., preferably at the temperature of 0-25° C., and most preferably at the temperature of 4° C.;

(2) Preparation of the Sustained-Release Drug Carrier

A. Drugs are added to the above hydrogel, dispersed and dissolved while stifling, and then a drug-loaded gelling agent is obtained through sterilization, aseptic filling and sealing; the gelling agent is preserved at the temperature of 0-50° C., preferably at the temperature of 0-25° C., and most preferably at the temperature of 4° C.; or a dried gelling agent is obtained by drying, and is then preserved at the temperature of −70° C.-50° C., preferably at the temperature of 0-25° C., and more preferably at the temperature of 4° C.;

B. Preparation with the dried gelling agent as the raw material: dried gelling agent is uniformly dispersed in aqueous solution while oscillating at the temperature of 0-300° C., preferably at the temperature of 0-70° C., and most preferably at the temperature of 0-25° C. Drugs are dispersed and dissolved in dispersed gel while stirring. A drug-loaded gelling agent is obtained through sterilization, aseptic filling and sealing, and is then preserved at the temperature of −70° C.-50, preferably at the temperature of 0-25, and more preferably at the temperature of 4° C.

(3) Preparation of the Vascular Embolization Gelling Agent Used for Sustained Release of Drugs

The solution used for dispersion in the step (1) is aqueous solution, normal saline solution or PBS (phosphate buffer solution) at pH 7.4, and the proportion of drug carrier material to the solution is 0.01%-50% W/V, and is preferably 0.5-50% W/V.

The particle size of gel obtained in the step (1) is 10 nm −150 μm, and is preferably 100 nm-50 μm.

The sterilization method in the step (1) or (2) is one of the following methods: \textcircled{1} filtration with a filter membrane of 0.45 μm; \textcircled{2} conventional autoclaving; \textcircled{3} low-temperature sterilization: first cool to −20° C. and keep at this temperature for more than 3 hours, then cool to −70° C. and keep at this temperature for more than 3 hours, subsequently heat to −20° C. and keep at this temperature for more than 3 hours, and then heat to 4° C. and keep at this temperature for more than 3 hours, followed by UV irradiation for more than 30min. This process is repeated 2-5 times.

Operation in the whole preparation process of the step (1) and (2) is under sterile conditions, and all reagents and instruments thereof are sterile.

The drugs in the present invention include any antitumor drugs suitable for preparation of drug delivery system (including but not limited to), and may be organic anticancer drugs, water-soluble anticancer drugs or water-insoluble anticancer drugs, including alkylating agents such as nitrogen mustard, chlorambucil, cyclophosphamide (CTX) and ifosfamide (IFO), nitrosoureas such as N-methyl-N-nitrosourea (MNU), ACNU, BCNU, CCNU and methyl CCNU, ethylenimines such as 2,4,6-triethylene melamine compound (TEM) and thiotepa, methane sulfonate such as busulfan, dacarbazine, procarbazine and HXM; antimetabolic drugs including thymidylate synthase inhibitors such as 5-fluorouracil (5-FU), ftorafur (FT-207), tegadifur (difuradin FD-1), tegafur-uracil (UFT) and furtulon (5-DFUR), dihydrofolate reductase inhibitors such as methotrexate (MTX) and andaminopterin, DNA polymerase inhibitors such as cytarabine (Ara-c), ribonucleotide reductase inhibitors such as hydroxyurea (HU), inosine dialdehyde, adenosinediialde-hgde and guanazole, purine nucleotide synthesis inhibitors such as 6-mercaptopurine (6-MP); antitumor antibiotics including anthracycline antitumor antibiotics such as adriamycin (ADM), daunorubicin (DNR), epirubicin (EPI or E-ADM), mitoxantrone (MTT, DHAD) and pirarubicin (THP), actinomycin antitumor antibiotics such as actinomycin D (ACD), bleomycin antitumor antibiotics such as bleomycin and pingyangmycin (A5), mitomycin antitumor antibiotics such as mitomycin A, mitomycin B and mitomycin C (MMC), mithramycin antitumor antibiotics such as mithramycin (MTH) and olivomycin, other antibiotics such as streptozotocin (STT); antitumor herbs: vincaleukoblastine and taxad inhibiting the polymerization of microtubule and tubulin such as vinblastine (VLB), vincristine (VCR), vindesine (VDS), navelbine (NVB), paclitaxel (PTX) and taxotere, topoisomerase inhibitor camptothecin and podophyllotoxin such as camptothecin (CPT), hydroxy camptothecin (HCPT) and etoposide (VP-16), antitumor drugs inhibiting DNA synthesis in tumor cells such as harringtonine and indirubin; other antitumor drugs such as cisplatin (DDP), carboplatin (CBP) and oxaliplatin (L-OHP).

The preferred drugs include epirubicin, cisplatin, vincristine, paclitaxel, docetaxel and etoposide.

The drugs in the present invention include any tumor angiogenesis inhibitors suitable for preparing gel micelle drug delivery system, such as the drugs inhibiting matrix degradation: marimastat, AG3340, COL-3, Bay 12-9556, BM S-275291 or neovastat; drugs directly acting on endothelial cells: TNP-470, squalamine, AE-941 or endostatin; drugs inhibiting angiogenesis factors: SU5416, SU6668, interferon a or anti-VEGF antibody; drugs inhibiting the integrin identification: vitaxin (endostatin) or EMD 121974; other nonspecific inhibitors such as thalidomide, CA I, interleukin 12, suramin or IM862.

The preferred drugs include vitaxin, interleukin 12 or endostatin.

The drugs in the present invention include any molecular targeted anti-tumor drugs suitable for preparing gel micelle drug delivery system, such as protein tyrosine kinase inhibitors: imatinib, erlotinib, sunitinib, gefitinib, sorafenib, dasatinib, lapatinib and nilotinib etc. The preferred drugs include imatinib, gefitinib and sorafenib.

The most preferable drug is endostatin.

The drugs in the present invention include developers, which may be micronized tantalum powder, tantalum oxide, barium sulfate, magnetic particle or iohexol. Where, iohexol is preferable.

The embolization agent in the present invention can be used for arterial embolism, achieve the effect of inhibiting tumor neovascularization through local sustained release, and be immediately solidified after reaching the target vessels, so as to quickly achieve thorough embolization and further improve the efficacy of TACE with less side reaction and no influence on the functions of heart, liver and kidney etc.

The embolization agent in the present invention is convenient for transcatheter injection, and can enter different tiny branches of vessels with the bloodstream. In the injection process, it is difficult to flow back, and it does not pass through the lateral anastomosis branches. The thermosensitive gel in the present invention can be used as not only the embolization agent of transcatheter arterial chemoembolization for various malignant tumors, but also the endovascular embolization agent for benign diseases such as arterial embolization treatment for hysteromyoma, pulmonary hemoptysis, hemorrhage of digestive tract or postpartum hemorrhage, and has high value in clinical application. The preparation method in the present invention is simple and suitable for industrialized production.

### BRIEF DESCRIPTION OF THE DRAWINGS

FIGS. 1a and 1b show the embolization effect in rabbits of Example 13. FIG. 1a shows the DSA hepatic arteriography before embolization; FIG. 1b shows the DSA hepatic arteriography after embolization with peripheral hepatic arterial embolization. (Example 18)

FIGS. 2a and 2b show the CT in 24 hour after embolization of experimental rabbits with intrahepatic deposition in Example 13 (Example 22)

FIGS. 3a and 3b show the CT in 48 hour after embolization of experimental rabbits with intrahepatic deposition in Example 13 (Example 22)

FIG. 4a shows the CT in 8 day after embolization of experimental rabbits with necrosis of the left lobe of liver and deposition of embolization agent on the edge in Example 13 (Example 23)

FIG. 4b shows the CT in 21 day after embolization of experimental rabbits with spotty necrosis of the left lobe of liver and deposition of embolization agent on the edge in Example 13 (Example 23)

FIGS. 5a and 5b show the DSA hepatic arteriography before and after embolization of experimental rabbits with occlusion of peripheral vessels after hepatic arterial embolization (Example 24)

FIG. 6 shows the reexamination CT in 24 hour after embolization of experimental rabbits with deposition of embolization agent in liver parenchyma in Example 14 (Example 25)

FIGS. 7a and 7b show the reexamination CT in 48 hour after embolization of experimental rabbits with deposition of embolization agent in liver parenchyma in Example 14 Example 25)

FIGS. 8A, 8B, and 8C show the reexamination CT in 24 and 48 hour after direct injection of embolization agent into liver parenchyma on the left lobe of liver in experimental rabbits with deposition of embolization agent in liver parenchyma, and that in one week without deposition of embolization agent. (Example 26)

FIGS. 9a and 9b show the gross pathological specimen after embolization (Example 22) FIG. 10A confirms intrahepatic tumor growth, FIG. 10B defines feeding arteries of the tumor, and the angiography in FIG. 10C shows complete embolization of feeding arteries of the tumor (Example 27)

FIGS. 11A, 11B, and 11C show the reexamination CT in 3 week after operation and histopathological examination after killing animals, showing complete necrosis status of the tumor. (Example 27)

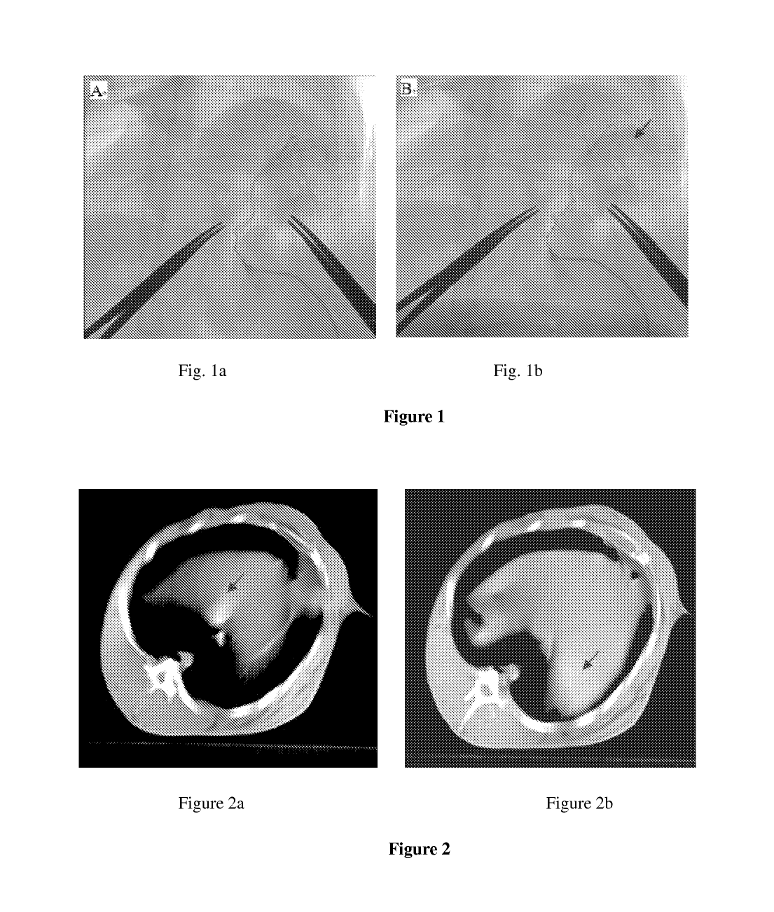

### DETAILED DESCRIPTION OF THE INVENTION

The present invention is further illustrated with the following examples, but is not limited to the following examples.

### EXAMPLE 1

### Purification of Poloxamer 407

Poloxamer 407 available on the market was exactly dissolved in appropriate amount of dichloromethane. The solution thus obtained was dropped to excess n-hexane while stifling, laid aside until complete precipitation, and then centrifuged. Poloxamer 407 was obtained by drying the precipitate under reduced pressure.

### EXAMPLE 2

### Purification of Poloxamer 407

Poloxamer 407 available on the market was exactly dissolved in appropriate amount of ethanol. The solution thus obtained was dropped to excess ethyl ether while stirring, laid aside until complete precipitation and then centrifuged. Poloxamer 407 was obtained by drying the precipitate through rotary evaporation under reduced pressure.

### EXAMPLE3

### Purification of Poloxamer 407

Poloxamer 407 available on the market was exactly dissolved in appropriate amount of water. The solution thus obtained was extracted with proper amount of n-propanol, and then laid aside until complete stratification. Poloxamer 407 was obtained by freeze drying of the aqueous layer.

### EXAMPLE 4

### Purification of Polyvinylpyrrolidone PVP K90

Polyvinylpyrrolidone PVP K90 available on the market was exactly dissolved in appropriate amount of ethanol. The solution thus obtained was dropped to n-hexane while stirring, laid aside until complete precipitation, and then centrifuged. Polyvinylpyrrolidone PVP K90 was obtained by drying the precipitate under reduced pressure.

### EXAMPLE 5

### Purification of Polyvinylpyrrolidone PVP K30

Polyvinylpyrrolidone PVP K30 available on the market was exactly dissolved in appropriate amount of dichloromethane. The solution thus obtained was dropped to ethyl ether while stifling, laid aside until complete precipitation, and then centrifuged. Polyvinylpyrrolidone PVP K30 was obtained by drying of the precipitate at 40° C.

### EXAMPLE 6

### Purification of Polyvinylpyrrolidone PVP K25

Polyvinylpyrrolidone PVP K25 available on the market was exactly dissolved in appropriate amount of ethyl acetate. The solution thus obtained was dropped to n-hexane while stirring, laid aside until complete precipitation, and then centrifuged. Polyvinylpyrrolidone PVP K25 was obtained by drying the precipitate under reduced pressure.

### EXAMPLE 7

### Preparation of the Gelling Agent:

10 g of poloxamer 407 was added to 40 ml of ultrapure water, and laid aside at 4° C. for 1 day to completely dissolve the polymer. The gel dispersion system thus obtained was sterilized with regular heat sterilization method, then sealed after aseptic filling and preserved at 4° C.

### EXAMPLE 8

### Preparation of the Freeze-Dried Gelling Agent

7.4 g of poloxamer 407 and 0.3 g of polyvinylpyrrolidone PVP K30 were added to 30 ml of ultrapure water, and laid aside at 4° C. for 3 days to completely dissolve the polymer. The gel dispersion system thus obtained was sealed after freeze drying, and then preserved at 4° C.

### EXAMPLE 9

### Preparation of the Gelling Agent

0.84 g of poloxamer 338 was added to 3 ml of ultrapure water, and laid aside at 4° C. for 24 h to completely dissolve the polymer. The gel dispersion system thus obtained was sealed after vacuum drying, and then preserved at 4° C.

### EXAMPLE 10

### Preparation of the Gelling Agent

7.4 g of poloxamer 407 and 0.2g of PEG-modified polylactic acid (PLA-PEG) were respectively added to 20 ml and 30 ml of ultrapure water, and laid aside at 25° C. for 3 days to completely dissolve the polymer. The gel dispersion system obtained through vortex oscillation of the mixed solution of both was sealed after evaporation of water under reduced pressure and drying, and then preserved at 4° C.

### EXAMPLE 11

### Preparation of the Freeze-Dried Gelling Agent

4.8 g of poloxamer 407 and 0.4 g of PEG-PLGA were respectively added to 20 ml and 10 ml of ultrapure water, and laid aside at 4° C. for 3 days to completely dissolve the polymer. The gel dispersion system obtained through vortex oscillation of the mixed solution of both was sealed after freeze drying, and then preserved at 4° C.

### EXAMPLE 12

### Preparation of the Gel System

23 g of poloxamer 407 and 2 g of poloxamer 188 were added to 51 ml of ultrapure water, and laid aside at 5° C. for 2 days to completely dissolve the polymer. A gel system of poloxamer 407 and poloxamer 188 can be obtained by fully stirring. The gel dispersion system thus obtained was sealed after filtration with 0.45 μm filter membrane and aseptic filling, and then preserved at 4° C.

### EXAMPLE 13

### Preparation (Aseptic Manipulation) of Gel Developer “Iohexol” Embolization Agent

The thermosensitive gel dispersion system containing the developer iohexol was prepared by dispersing 1.76 g of the hydrogel prepared in Example 8 and 1.5 mg of iohexol in 4 ml of ultrapure water present in a sterile reagent bottle while oscillating. The system thus obtained was sterilized, sealed, and then preserved in a refrigerator at 4° C.

Sterilization method: The prepared gel was first cooled to −20° C. and kept at this temperature for 24 h, then cooled to −70° C. and kept at this temperature for 12 h, subsequently slowly heated to −20° C. and kept at this temperature for 10 h, and then heated to 4° C. and kept at this temperature for 24 h, followed by UV irradiation for 6 h. This process was repeated 3 times.

### EXAMPLE 14

### Preparation (Aseptic Manipulation) of Gel Developer “Iohexol” (2) Embolization Agent

The thermosensitive gel embolization agent containing the developer iohexol was prepared by dispersing 1.54 g of the freeze-dried gelling agent prepared in Example 8 and 1 mg of iohexol in 6 ml of ultrapure water present in a sterile reagent bottle while oscillating and then laying aside the system thus obtained at room temperature of 25° C. for 2 days. The system was sterilized, sealed, and then preserved in a refrigerator at 4° C.

Sterilization method: The prepared gel was first cooled to −20° C. and kept at this temperature for 24 h, then cooled to −70° C. and kept at this temperature for 12 h, subsequently heated to −20° C. and kept at this temperature for 10 h, and then heated to 4° C. and kept at this temperature for 6 h, followed by UV irradiation for 1 h. This process was repeated 3 times. The system thus obtained was sealed, and then preserved in a refrigerator at 4° C.

### EXAMPLE 15

### Preparation (Aseptic Manipulation) of Recombinant Human Endostatin Embolization Agent

0.457 g of freeze-dried gelling agent prepared in Example 8 and 15 mg of recombinant human endostatin were dispersed in 3 ml of ultrapure water present in a sterile reagent bottle while oscillating, and then laid aside at 4° C. overnight to prepare the gel embolization agent carrying recombinant human endostatin. The recombinant human endostatin thus obtained was first cooled to −20° C. and kept at this temperature for 48h, then cooled to −70° C. and kept at this temperature for 12 h, subsequently heated to −20° C. and kept at this temperature for 24 h, and then heated to 4° C. and kept at this temperature for 24 hours, followed by UV irradiation for 2 hours. This process was repeated twice. The recombinant human endostatin was sealed, and then preserved in a refrigerator at 4° C.

### EXAMPLE 16

### Preparation (Aseptic Manipulation) of Recombinant Human Endostatin Embolization Agent

2.11 g of freeze-dried gelling agent prepared in Example 8 and 25 mg of recombinant human endostatin were ultrasonically dispersed in 5 ml of ultrapure water present in a sterile reagent bottle while oscillating, and then laid aside at 4° C. overnight to prepare the recombinant human endostatin embolization agent. Afterwards, the recombinant human endostatin thus obtained was first cooled to −20° C. and kept at this temperature for 48 h, then cooled to −70° C. and kept at this temperature for 12 h, subsequently heated to −20° C. and kept at this temperature for 24 h, and then heated to 4° C. and kept at this temperature for 24 h, followed by UV irradiation for 3 h. This process was repeated twice. After aseptic filling, the recombinant human endostatin was sealed, and then preserved in an environment at 4° C. The formulated gel can become stable in an environment at 37° C. within 4 min.

### EXAMPLE 17

### Preparation (Aseptic Manipulation) of Epirubicin Embolization Agent

2.22 g of poloxamer 407, 70 mg of PVPK-30 and 10 mg of epirubicin hydrochloride were simultaneously added to a sterile reagent bottle, and then exposed to ultraviolet irradiation for 4 h. In a ultraclean bench, 9 ml of ultrapure water was added to the reagent bottle, which was then laid aside at 4° C. for 3 days, and dispersed by oscillation, so that the epirubicin embolization agent was obtained. The prepared epirubicin embolization agent was first cooled to −20° C. and kept at this temperature for 48 h, then cooled to −70° C. and kept at this temperature for 24 h, subsequently heated to −20° C. and kept at this temperature for 24 h, and then heated to 4° C. and kept at this temperature for 24 h, followed by UV irradiation for 3 h. This process was repeated twice. After aseptic filling, the embolization agent was sealed and then preserved in a refrigerator at 4° C. It can be used to treat bladder cancer.

### EXAMPLE 18

### Preparation (Aseptic Manipulation) of Imatini Embolization Agent

2.22 g of poloxamer 407, 70 mg of PVPK-30 and 10 mg of imatinib mesylate were simultaneously added to a sterile reagent bottle, and then exposed to ultraviolet irradiation for 4 h. In a ultraclean bench, 9 ml of ultrapure water was added to the reagent bottle, which was then laid aside at 4° C. for 3 days, and dispersed by oscillation, so that the imatinib mesylate embolization agent was obtained. The imatinib mesylate embolization agent thus obtained was first cooled to −20° C. and kept at this temperature for 48 h, then cooled to −70° C. and kept at this temperature for 24 h, subsequently heated to −20° C. and kept at this temperature for 24 h, and then heated to 4° C. and kept at this temperature for 24 h, followed by UV irradiation for 3 h. This process was repeated twice. After aseptic filling, the embolization agent was sealed and then preserved in a refrigerator at 4° C. . It can be used to treat small cell lung cancer.

### EXAMPLE 19

### Preparation (Aseptic Manipulation) of Oxaliplatin Embolization Agent

2.5 g of poloxamer 407, 50 mg of PVPK-30 and 20 mg of oxaliplatin were simultaneously added to a sterile reagent bottle, and then exposed to ultraviolet irradiation for 4 h. In a ultraclean bench, 10 ml of ultrapure water was added to the reagent bottle, which was then laid aside at 4° C. for 3 days, and dispersed by oscillation, so that the oxaliplatin embolization agent was obtained. The oxaliplatin embolization agent thus obtained was first cooled to −20° C. and kept at this temperature for 48 h, then cooled to −70° C. and kept at this temperature for 24 h, subsequently heated to −20° C. and kept at this temperature for 24 h, and then heated to 4° C. and kept at this temperature for 24 h, followed by UV irradiation for 3 h. This process was repeated twice. After aseptic filling, the embolization agent was sealed and then preserved in a refrigerator at 4° C. It can be used to treat colorectal cancer.

### EXAMPLE 20

### Preparation (Aseptic Manipulation) of Cisplatin Embolization Agent

2.3 g of poloxamer 407, 20 mg of PVPK-30 and 10 mg of cisplatin were simultaneously added to a sterile reagent bottle, and then exposed to ultraviolet irradiation for 4 h. In a ultraclean bench, 10 ml of ultrapure water was added to the reagent bottle, which was then laid aside at 4° C. for 3 days, and dispersed by oscillation, so that the cisplatin embolization agent was obtained. The cisplatin embolization agent thus obtained was first cooled to −20° C. and kept at this temperature for 48 h, then cooled to −70° C. and kept at this temperature for 24 h, subsequently heated to −20° C. and kept at this temperature for 24 h, and then heated to 4° C. and kept at this temperature for 24 h, followed by UV irradiation for 3 h. This process was repeated twice. After aseptic filling, the embolization agent was sealed and then preserved in a refrigerator at 4° C. It can be used to treat cervical cancer.

### EXAMPLE 21

### Preparation (Aseptic Manipulation) of Sorafenib Embolization Agent

0.22g of poloxamer 407, 70 mg of PVPK-30 and 10 mg of sorafenib were simultaneously added to a sterile reagent bottle, and then exposed to ultraviolet irradiation for 4 h. In a ultraclean bench, 9 ml of ultrapure water was added to the reagent bottle, which was then laid aside at 4° C. for 3 days, and dispersed by oscillation, so that the sorafenib embolization agent was obtained. The sorafenib embolization agent thus obtained was first cooled to −20° C. and kept at this temperature for 48 h, then cooled to −70° C. and kept at this temperature for 24 h, subsequently heated to −20° C. and kept at this temperature for 24 h, and then heated to 4° C. and kept at this temperature for 24 h, followed by UV irradiation for 3 h. This process was repeated twice. After aseptic filling, the embolization agent was sealed and then preserved in a refrigerator at 4° C. It can be used to treat renal cancer.

### EXAMPLE 22

### Hepatic Arterial Embolization in Healthy Experimental Rabbits

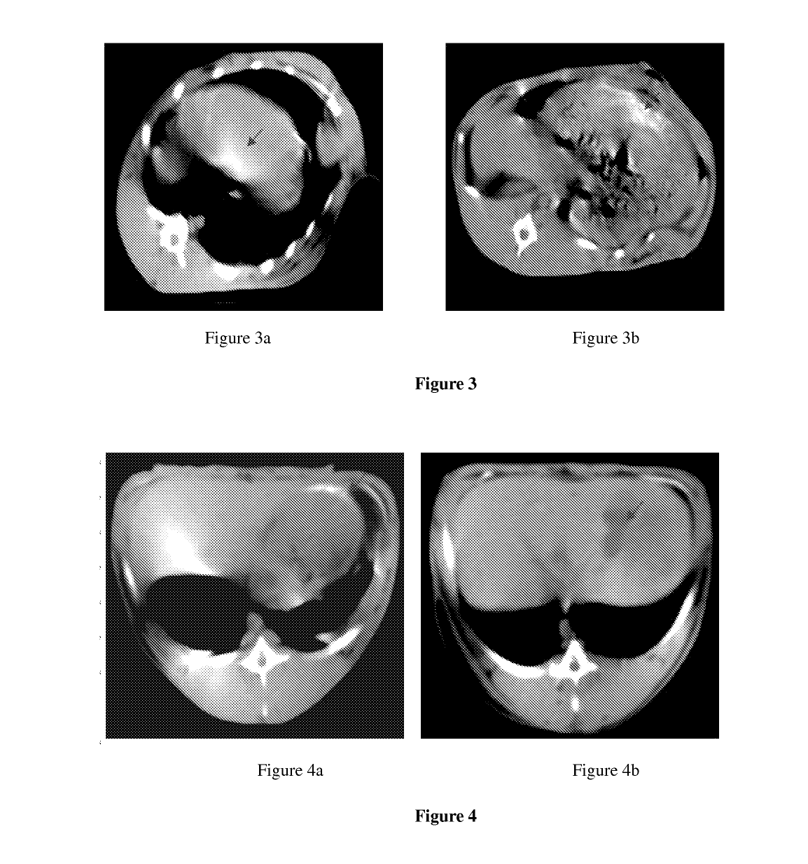

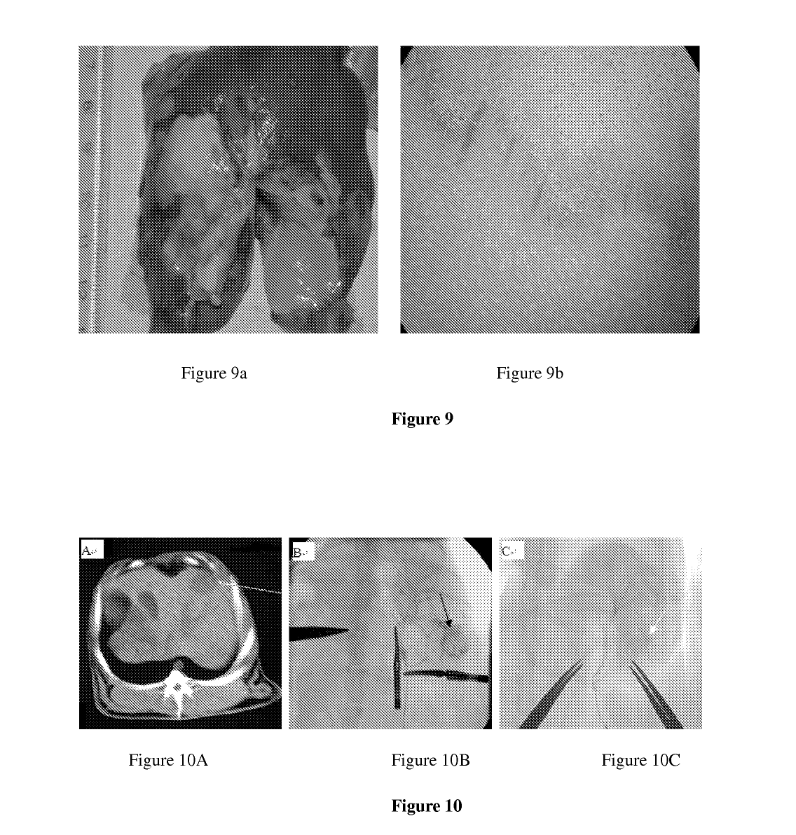

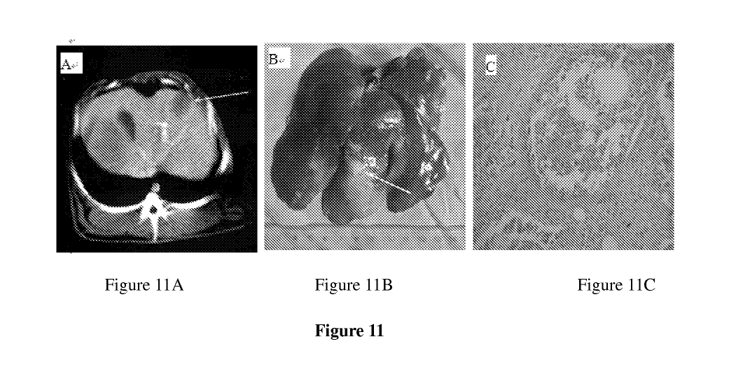

Under conventional preparation, ketamine hydrochloride (3.5-4 mg/kg) was intravenously injected to the ear edge of rabbit for intravenous anesthesia. Subsequently, the rabbit was laparotomized, and its stomach was turned outwards to the left side to expose its hepatic portal region. Its common hepatic artery was isolated along the inner side of the portal vein, and was distally ligated with 2 ligation lines respectively at the upper end and lower end. After its proximal end was pulled up to stop bleeding, a small opening was cut on its artery to insert a 24G vein detained flexible pipe into the proper hepatic artery, to determine its hepatic artery and branch developing through DSA angiography, followed by transcatheter injection of 1.5 ml of self-made “iohexol” embolization agent (Example 13). After reexamination angiography showed complete embolization of tiny branches downstream the hepatic artery segment, the pipe was pulled out, and its abdomen was closed to complete the laparotomy. CT reexamination was respectively carried out in 24 and 48 h after the operation, and the animals were killed in 48 h for gross liver specimen and pathological examination. CT results (FIGS. 2, 3) showed: in 24-48 hours, embolization agent was scattered in the liver parenchyma of the embolization area, which was especially obvious in the left lobe; (FIG. 9) gross specimen showed that there was patchy ischemic necrosis on the edge of the left lobe of the liver, and the pathology showed 80% necrosis of hepatocytes.

### EXAMPLE 23

### Hepatic Arterial Embolization in Healthy Experimental Rabbits

Implementation of the operation was similar to that in Example 18. 2.5 ml of embolization agent (Example 13) was injected into the hepatic artery. The reexamination DSA angiography showed complete embolization of the tiny branches downstream the left lobe of the liver. CT reexamination in 24 and 48 h showed embolization agent deposition in the liver parenchyma. The reexamination in 1 week after the operation showed spherical necrosis of the left lateral lobe of liver, and embolization agent deposition on the edge of the necrosis area (FIG. 4).

### EXAMPLE 24

### Hepatic Arterial Embolization in Healthy Experimental Rabbits

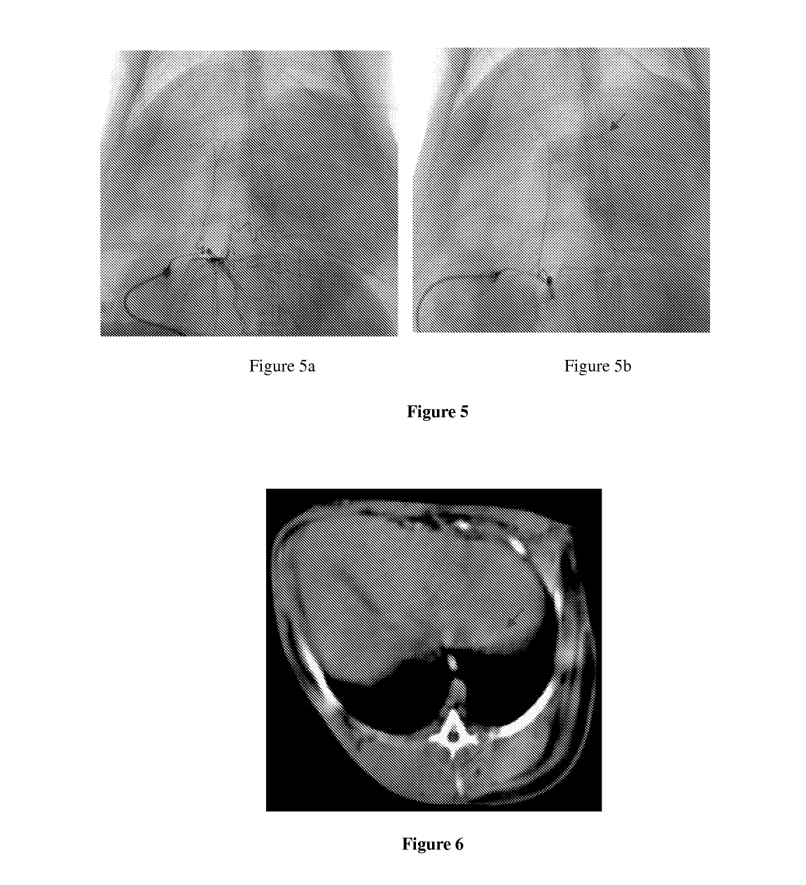

Implementation of the operation was similar to that in Example 18. 2.5 ml of embolization agent (Example 14) was injected into the hepatic artery. The reexamination DSA angiography showed complete embolization of the tiny branches downstream the left lobe of the liver. CT reexamination in 24 and 48 h showed embolization agent deposition in the liver parenchyma (FIG. 5).

### EXAMPLE 25

### Hepatic Arterial Embolization in Healthy Experimental Rabbits

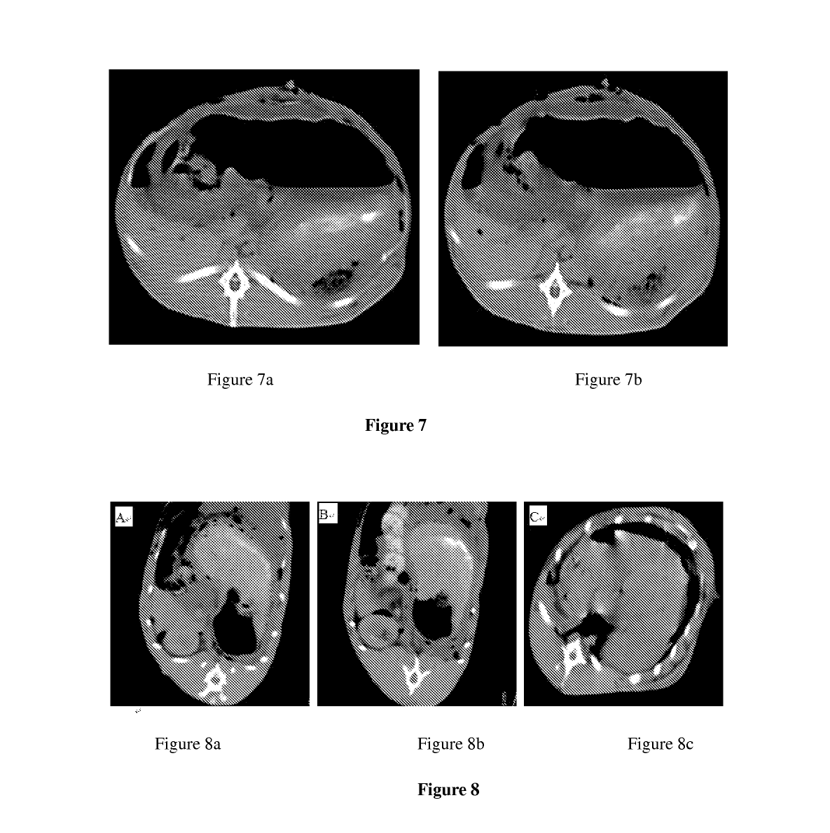

Under conventional preparation, ketamine hydrochloride (3.5-4 mg/kg) was intravenously injected into the ear edge of rabbit for intravenous anesthesia. Subsequently, the rabbit was laparotomized, and its stomach was turned outwards to the left side to expose its hepatic portal region. Its common hepatic artery was isolated along the inner side of the portal vein, and was distally ligated with 2 ligation lines respectively at the upper end and lower end. After its proximal end was pulled up to stop bleeding, a small opening was cut on its artery to insert a 24G vein detained flexible pipe into the proper hepatic artery, to determine its hepatic artery and branch developing through DSA angiography, followed by transcatheter injection of 1.5 ml of self-made embolization agent (Example 14). After reexamination angiography showed complete embolization of tiny branches downstream the hepatic artery segment, the pipe was pulled out, and the abdomen was closed to complete the laparotomy. CT reexamination (FIGS. 6, 7) was respectively carried out in 24 and 48 h after operation, and the animals were killed in 48 h for gross liver specimen and pathological examination. CT results showed: in 24-48 hours, embolization agent was scattered in the liver parenchyma of the embolization area.

### EXAMPLE 26

### Direct Injection into the Liver Parenchyma of Healthy Experimental Rabbits

Animal anesthesia and laparotomy were similar to the above description. After laparotomy, the left lobe of liver was exposed, and 2 ml of embolization agent (Example 14) was directly injected into the left lobe of liver. Reexamination CT was carried out respectively in 24 h, 48 h and 1 week after the laparotomy. The CT in 48 h showed embolization agent deposition in the left lobe of liver, and that in 1 week did not show embolization agent in the liver parenchyma. (FIG. 8: A, B, C)

### EXAMPLE 27

### Interventional Therapy of Transplanted Liver Cancer in Rabbits

After laparotomy, the VX-II tumor strain of about 1 cm³ was planted directly in the left lobe of liver of rabbits. 2 weeks later, tumor growth in the liver was determined through the reexamination CT (FIG. 10A). Afterwards, the rabbits were laparotomized again, and the feeding arteries of tumor were determined through hepatic artery DSA angiography (the same method as that in above description) (FIG. 10B). Then, about 2 ml of thermosensitive gel embolization agent “endostatin—15 mg” (Example 15) was injected into the hepatic artery, and the operation was completed after the reexamination angiography showed complete embolization of the feeding arteries of tumor (FIG. 10C). In 24 h, 48 h and 1, 2, 3 week after operation, blood was respectively drawn for blood routine examination and liver and kidney function examination to understand the toxic reaction and side effect after embolization; the size and necrosis of liver tumor was observed through CT reexamination, and the blood supply of tumor and necrosis situation were observed through reexamination DSA in 3 week after operation and histopathological examination after killing the animals. (as shown in FIG. 11: A, B, C)

### Conclusions:

1. Hepatic arterial embolization in healthy experimental rabbits: The thermosensitive embolization agent “iohexol” can be directly injected through a microcatheter with the inner diameter of 0.75 mm at normal temperature. After injection, the embolization agent can selectively enter peripheral vessels of the hepatic arteries, be rapidly solidified, and form tiny peripheral arterial embolization.

2. After embolization, there was local deposition of intrahepatic “iohexol” developer for 4 weeks.

3. 1 week after embolization, there was significant local necrosis of liver tissues. The pathological situation 4 weeks later showed that the liver lobe structure disappeared on the embolized site, and hepatocytes were completely necrotic and had fibrillar connective tissues.

4. After embolization, there was neither severe toxic reaction nor severe side effect except for transient increase of the liver function (AST, ALT).

5. The transplanted liver cancer treatment experiment confirmed that: the group treated with thermosensitive embolization agent “endostatin” had best efficacy, followed by the group treated with thermosensitive embolization agent “iohexol”. The group perfused with “endostatin” only was weakly better than the control group.

Therefore, the thermosensitive embolization agent “endostatin” in the present invention has the following advantages: this embolization agent is “thermosensitive”, and its liquid at normal temperature is convenient for transcatheter injection; it is rapidly solidified in vivo, and can achieve the effect of vascular embolization; after embolization, it has neither severe toxic reaction nor severe side effects and its efficacy is reliable; during the embolization, it can achieve local sustained release of drugs in tumor, inhibit tumor angiogenesis, and achieve the purpose of dual treatment; it is a desired vascular embolization agent.

We claim:

1. A vascular embolization gelling agent for sustained release of drugs for treating tumors, comprising
a drug in an amount of about 0.01% to 80% of the agent, and
an encapsulation for the drug in a form of a gel,
wherein the gel is made of a drug carrier.

2. The vascular embolization gelling agent as defined in claim 1, wherein the drug is in the amount of about 0.05% to 50% of the agent.

3. The vascular embolization gelling agent as defined in claim 1, wherein mass percentage of the drug carrier in the agent is about 10%-65%.

4. The vascular embolization gelling agent as defined in claim 1, wherein particle size of the gel is in a range of about 10 nm to 150 μm.

5. The vascular embolization gelling agent as defined in claim 4, wherein the particle size of the gel is in the range of about 100 nm to 50 μm.

6. The vascular embolization gelling agent as defined in claim 1, wherein the drug carrier is polymer poloxamer, polyvinylpyrrolidone, polyethylene glycol, polyvinyl alcohol, carbomer, polymethacrylates, PEG-modified polylactic acid, PEG-modified polylactic acid-glycolic acid, PEG-modified polyglycolic acid, PEG-modified polycaprolactone, methyl cellulose, ethyl cellulose, hydroxyethyl cellulose, hydroxyethyl methyl cellulose, hydoxymethyl cellulose, hydroxypropyl methyl cellulose, hydroxypropyl cellulose, carboxymethyl cellulose, sodium carboxymethyl cellulose; pregelatinized starch, gum acacia, tragacanth gum, carrageenan, locust bean gum, guar gum, konjac gum, alginate, gelatin, hyaluronic acid, agar, chitosan, deacetylized chitosan or galactomannan, cyclodextrin, cyclodextrin derivatives, or a mixture thereof.

7. The vascular embolization gelling agent as defined in claim 6, wherein molecular weight of each polymer in the drug carrier is about 0.1K to 5000K Dalton.

8. The vascular embolization gelling agent as defined in claim 7, wherein the molecular weight of each polymer in the drug carrier is about 0.5K-50K Dalton.

9. The vascular embolization gelling agent as defined in claim 6, wherein the drug carrier comprises the poloxamer, optionally with one or more of the other polymers.

10. The vascular embolization gelling agent as defined in claim 6, wherein the drug carrier is a combination of poloxamer 407 with one or more of other polymers, and the poloxamer 407 is in an amount of 0.1% to 100% of the drug carrier.

11. The vascular embolization gelling agent as defined in claim 10, wherein the other polymer is polyvinylpyrrolidone.

12. The vascular embolization gelling agent as defined in claim 1, wherein the drug carrier is purified before use in the agent.

13. The method for purifying the drug carrier as defined in claim 1, comprising
dissolving the drug carrier in a solvent that is methanol, ethanol, isopropanol, chloroform, dichloromethane, ethyl acetate, water, or a mixture thereof to form a solution, and
precipitating the solution in an ether or an alkane.

14. The method for purifying the drug carrier as defined in claim 13, wherein the solution is precipitated in ethyl ether or n-hexane.

15. The method for purifying the drug carrier as defined in claim 1, comprising
lay separating the drug carrier in an n-propanol/water solvent, and
drying the separated drug carrier.

16. The vascular embolization gelling agent as defined in claim 1, wherein the drug is nitrogen mustard, chlorambucil, cyclophosphamide, ifosfamide, N-methyl-N-nitrosourea, ACNU, BCNU, CCNU, methyl CCNU, 2,4,6-triethylene melamine compound, thiotepa, busulfan, dacarbazine, procarbazine, HXM, fluorouracil, ftorafur, tegadifur, tegafur-uracil, furtulon, methotrexate, andaminopterin, cytarabine, hydroxyurea, inosine dialdehyde, adenosinediialde-hgde, guanazole, 6-mercaptopurine, adriamycin, daunorubicin, epirubicin, mitoxantrone, pirarubicin, actinomycin D, bleomycin, pingyangmycin, mitomycin A, mitomycin B, mitomycin C, mithramycin, olivomycin, streptozotocin, vinblastine, vincristine, vindesine, navelbine, paclitaxel, taxotere, camptothecin, hydroxy camptothecin, etoposide, harringtonine, indirubin, cisplatin, carboplatin, oxaliplatin, imatinib, erlotinib, sunitinib, gefitinib, sorafenib, dasatinib, lapatinib, nilotinib, marimastat, AG3340, COL-3, Bay 12-9556, BM S-275291, neovastat, TNP-470, squalamine, AE-941, endostatin, SU5416, SU6668, interferon α, anti-VEGF antibody, vitaxin, EMD 121974, thalidomide, CA I, interleukin 12, suramin, or IM8624.

17. The vascular embolization gelling agent as defined in claim 1, wherein the drug is endostatin, epirubicin, oxaliplatin, imatinib, sorafenib, cisplatin, vincristine, etoposide, vitaxin, interleukin 12, or endostatin.

18. The vascular embolization gelling agent as defined in claim 1, wherein the drug is micronized tantalum powder, tantalum oxide, barium sulfate, magnetic particle, or iohexol.

19. The vascular embolization gelling agent as defined in claim 1, wherein the drug is iohexol.

20. A method for preparing the vascular embolization gelling agent for sustained release of drugs, comprising
(1) Preparing a gelling agent
One or a mixture of more of the drug carriers are dispersed in the solution while oscillating, and then laid aside at the temperature of 0-300° C., preferably at the temperature of 0-70° C., and most preferably at the temperature of 0-25° C., until the carrier materials are completely swelled, and dispersed and dissolved into a transparent liquid while stirring. Subsequently, a liquid gelling agent can be obtained by sterilization, aseptic filling and sealing; or a dried gelling agent is obtained through drying, and is preserved at the temperature of −70° C.-50° C., preferably at the temperature of 0-25° C., and most preferably at the temperature of 4° C. ;
(2) Preparing a sustained-release drug carrier
A. Drugs are added to the above liquid gel, dispersed and dissolved while stifling, and then a drug-loaded gelling agent is obtained through sterilization, aseptic filling and sealing; the gelling agent is preserved at the temperature of 0-50° C., preferably at the temperature of 0-25° C., and most preferably at the temperature of 4° C.;
B. The dried gelling agent obtained in step (1) is uniformly dispersed in aqueous solution while oscillating at the temperature of 0-300° C., preferably at the temperature of 0-70° C., and most preferably at the temperature of 0-25° C. Drugs are dispersed and dissolved in dispersed gel while stifling. A drug-loaded gelling agent is obtained through sterilization, aseptic filling and sealing, and is then preserved at the temperature of −70° C. -50° C., preferably at the temperature of 0-25 , and more preferably at the temperature of 4° C.

21. The preparation method as defined in claim 20, wherein the solution used for dispersion in step (1) is aqueous solution, normal saline solution or phosphate buffer solution (PBS) at pH 7.4; the proportion of polymer to the solution is 1:10-1:50 w/w; particle size of the obtained gel is 10 nm-150 μm, and is preferably 100 nm-50 μM.

22. The preparation method as defined in claim 20, characterized in that, the sterilization method in step (1) or (2) is one of the following methods: \textcircled{1} filtration with a filter membrane of 0.45 μm; \textcircled{2} conventional autoclaving; \textcircled{3} low-temperature sterilization: first cool to −20° C. and keep at this temperature for 1-48 hours, then cool to −70° C. and keep at this temperature for 1-48 hours, subsequently heat to −20° C. and keep at this temperature for 1-48 hours, and then heat to 4° C. and keep at this temperature for 1-48 hours, followed by UV irradiation for 30 min-24 h. This process is repeated 2-5 times.

23. The preparation method as defined in claim 20, wherein the drying method in step (1) or (2) may be one or more of rotary evaporation under reduced pressure, drying under reduced pressure, vacuum drying, freeze drying, spray drying, fluidized bed granulation drying and heat drying.

24. The vascular embolization agent as defined in claim 1, wherein the vascular embolization agent is used for transcatheter arterial chemoembolization for malignant tumors and arterial embolization for hysteromyoma, pulmonary hemoptysis, hemorrhage of digestive tract or postpartum hemorrhage.
